(19) United States
(12) Patent Application Publication
(10) Pub. No.: US 2017/0367637 A1
(43) Pub. Date: Dec. 28, 2017

(54) APPARATUS AND METHOD FOR MONITORING, ASSESSING AND MANAGING FUNCTIONAL STATE

(71) Applicant: Varicardstar, LLC, Eugene, OR (US)

(72) Inventors: Valeriy Vladimirovich STARODUBTSEV, Eugene, OR (US); Iurii Nikolayevich Semenov, Ryazan (RU)

(73) Assignee: Varicardstar, LLC

(21) Appl. No.: 15/195,281

(22) Filed: Jun. 28, 2016

(51) Int. Cl. A61B 5/22; A61B 5/0205; G06F 17/18

(52) U.S. Cl. CPC A61B 5/222; A61B 5/0205; G06F 17/18

(57) ABSTRACT

An apparatus and method for monitoring, assessing and managing an individual's functional state to allow the individual to reach and maintain optimized functional state based on customized guidance using as few as two to three functional state tests.

### CROSS-REFERENCE TO RELATED APPLICATIONS

This is an original U.S. patent application.

### FIELD OF INVENTION

This invention relates to non-invasive and integrated management of human physical condition.

### BACKGROUND

Human bodies are known to have the capacity or functional reserve to adapt to stress, whether externally from the surrounding environment or internally. A human body with high level of functional reserve is able to carry out extraordinary functional actions which generally is not possible for a human body with low level of functional reserve. A human body with high level of functional reserve is also able to better defend illnesses, diseases, and to slow the aging process. Thus, for purposes of maintaining a healthy functional state and increasing life span of a human body, it is helpful and critical to routinely monitor and assess the human body's functional reserve.

Various existing technologies allow a person's functional state to be monitored and assessed by one or more tests, including heart rate variability test, differential ECG test, brain wave test, jump test and stimulus response test. To achieve a relatively comprehensive assessment of functional state, the existing art prefers all five tests, which is cumbersome when the tests are conducted routinely. Thus, it is desirable to develop a new art that can reduce the number of preferred tests and to still achieve the goal of a relatively comprehensive understanding of functional state. One test that is yet to be utilized is anaerobic capacity test which has been researched and demonstrated to provide more insightful information of a person's functional reserve.

Additionally, although existing arts offer monitoring and assessment of a person's functional state which provide an understanding of the person's functional reserve, they do not provide specific guidance as to what exact steps need to be taken to improve functional reserve in order to achieve the goal of maintaining a healthy functional state, preventing diseases, and lengthening life span. A need exists to provide an individual with customized and specific guidance in addition to monitoring and assessment, which is based on the individual's specific functional state and allows the individual to easily follow in order to achieve or maintain a higher level of functional reserve.

### SUMMARY

The present invention utilizes anaerobic capacity test in combination with heart rate variability test and oxygen saturation level test to provide a relatively comprehensive understanding of an individual's functional reserve. By reducing the number of preferred tests from prior arts, the present invention makes it less burdensome for individuals to routinely monitor and assess functional reserve. With fewer required or preferred tests, it also cuts down manufacturing costs and thus saves consumers' expenses, without reducing the quality of assessment that is intended to achieve.

The present invention also offers integrated management to, improve functional reserve by not only providing customized guidance in combination with monitoring and assessment results, but also offering the built-in breathing exercise while an individual is performing anaerobic capacity test, which allows the individual to receive information of his or her functional state while simultaneously improving his or her functional state.

Because human bodies' physical condition may change at different times during the day, such integrated monitoring, assessment and management not only provide important information and guidance to individuals who intend to maintain and improve their health, but also to competitive athletes to help them understand their own bodies' physical status in order to better target training and competition achievements.

The present invention will be offered commercially that includes an apparatus with instructions either in the format of a CD ROM or an Internet link for downloading.

### BRIEF DESCRIPTION OF THE DRAWINGS

FIG. 1 is a perspective view of an embodiment of the invention, showing a data collector having a plurality of sensors for physical state tests, and a non-transitory computer-readable medium connected to the data collectors via a USB cable.

FIG. 2 is a focused view of an embodiment of the invention, showing a data collector having a plurality of sensors.

FIG. 3 is a representative output of the invention including, a plurality of graphical and textual components generated with data input from heart rate variability test, oxygen saturation level test, and anaerobic capacity test.

FIG. 4 is another representative output of the invention generated with data input from heart rate variability test and oxygen saturation level test, but without anaerobic capacity test.

### DETAILED DESCRIPTION

The present invention offers easy, low cost and non-invasive monitoring, assessment and management of functional reserve of a subject under test (SUT).

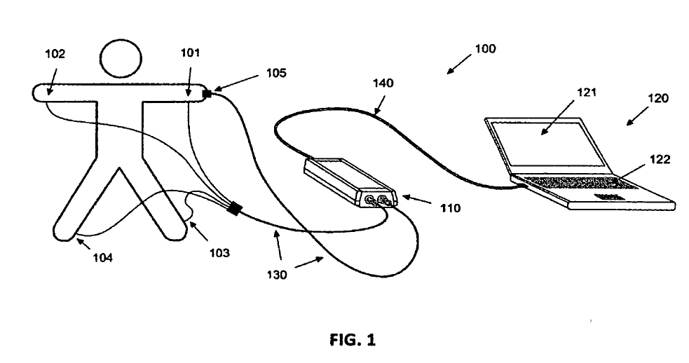

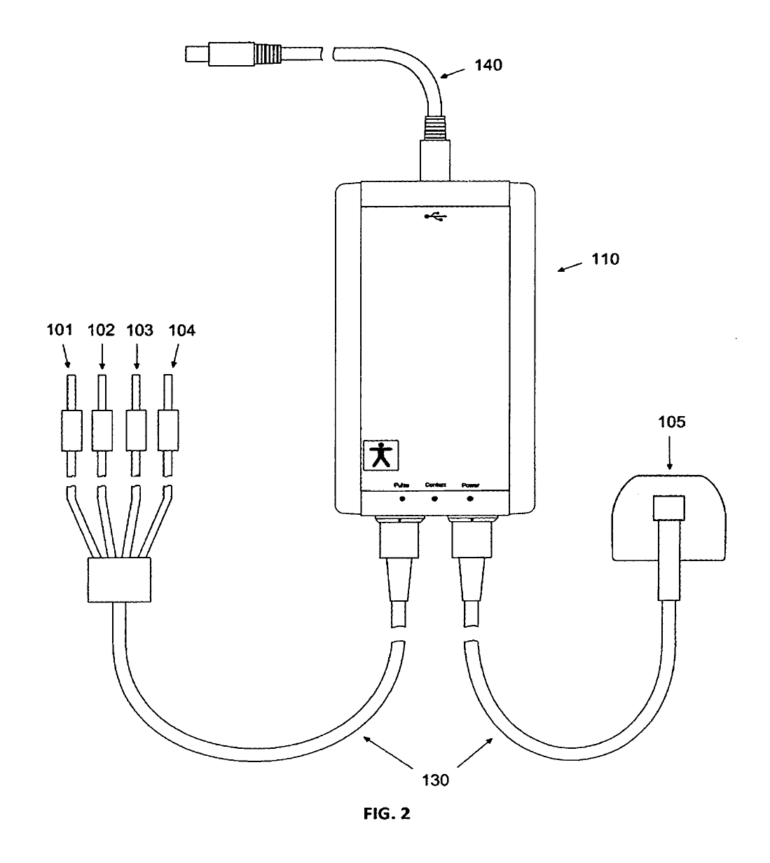

Referring to FIG. 1, the apparatus 100 includes a data collector 110 having sensors 101-105 for physical state tests performed on an SUT. Sensors 101-104 represent electrodes for heart rate variability (HRV) test that are preferably placed on each of the wrists and ankles of the SUT. Sensor 105 may be an oximeter for measurement of oxygen saturation of blood. If desired, additional sensors may be added for additional physical state tests, such as orthostatic test, which detects how human bodies adapt to changes of body position. The data collector 110 may be powered by batteries or a plug-in charger. Preferably, the data collector 110 amplifies, filters and digitizes analog signals from the sensors and propagates signals to a non-transitory computer-readable medium 120. The sensors may be connected to the data collector 110 via data cables 130, FIG. 2 gives a focused view centered at the data collector 110.

The non-transitory computer-readable medium 120, represented by a personal computer in FIG. 1, receives data from the data collector 110 via a USB cable 140 (as shown in FIG. 1) or via wireless Internet (such as Bluetooth or Wi-Fi or other forms of wireless connection). The computer-readable medium 120 has a user interface 121 and a data receiver 122. The data receiver 122 receives data collected from an anaerobic capacity test, which is performed separately from those performed by the sensors (but may be simultaneously collected). The data receiver 122 may be a keyboard to enter data.

The non-transitory computer-readable medium 120 further contains instructions which cause a programmable processor to read data propagated by the data collector 110, receive data from the data receiver 122 (data of the anaerobic capacity test), process all data, and display processing results with a plurality of graphical and textual output on the user interface 121. An individual will reach the most preferable result of improving his or her functional state by performing the anaerobic capacity test each time the HRV test and oxygen saturation level test are performed, because each anaerobic capacity test is also a training opportunity in addition to obtaining information of one's anaerobic capacity. The more anaerobic capacity test an individual performs routinely, the higher functional reserve the individual will develop over time. Although it is preferable to include data of the anaerobic capacity test, the present invention may function without the anaerobic capacity test, and results generated are not affected with respect to the functional reserve that is currently being monitored. In other words, if an SUT is simultaneously tested on two sets of the apparatus 100, one with the anaerobic capacity test and the other without the anaerobic capacity test, the assessment results and customized guidance will be the same. However, over time, the SUT's functional reserve will decline if the SUI consistently does not perform the anaerobic capacity test, compared to if the SUT routinely performs the anaerobic capacity test.

The flexibility of a built-in anaerobic capacity test or a breathing exercise is important and useful. An individual may choose to engage in the breathing exercise, i.e., perform the anaerobic capacity test, each time he or she monitors his or her functional state, without the need to set aside extra time to engage in breathing exercise to improve functional reserve. However, when performing anaerobic capacity test is challenging in certain circumstances, for example, when the SUT is in a very poor physical state due to injury, the SUT may continue to monitor his or her functional reserve without having to perform the anaerobic capacity test, which still allows the SUT an opportunity to follow the customized guidance to improve his or her functional reserve, even if slowly.

Data collected from the anaerobic capacity test is the amount of time passed while the SUT holds breath, and it may also include time passed while inhaling and exhaling as explained below. When performing the anaerobic capacity test, the SUT may choose to be tested while at rest or during physical activity, such as walking on a treadmill. Because of the flexibility of performing the anaerobic capacity test separately, the SUT may perform the HRV test and the oxygen saturation level test while at rest, and then perform the anaerobic capacity test while walking on a treadmill or doing other physical activities.

To perform the anaerobic capacity test, it is preferable to measure time passed at several different breathing phases, but it is not required to measure all phases. One phase for measurement is to measure maximum length of time passed while the SUT holds breath after inhaling, which begins immediately following a completion of inhalation (Maximum Hypoxic Time After Inhaling, or MHTAI). Another phase for measurement is to measure maximum length of time passed while the SUT holds breath beginning immediately following a completion of exhalation (Maximum Hypoxic Time After Exhaling, or MHTAE). Yet another phase for measurement is to measure maximum length of time passed for a complete breathing cycle, from the beginning of inhalation to the end of exhalation, which may include time passed while the SUT holds breath between inhalation and exhalation (Maximum Hypoxic Time for Inhale and Exhale, or MHTIE). For any phase of measurement, the SUT may first take a number of deep breaths before performing the test for time measurement.

Because an individual's physical condition can change even within 30 minutes, if an athlete is preparing for a competitive sports event and wishes to understand how his or her physical state changes during a day in order that he or she can train to reach an optimal physical state during the desired time, it is desirable for the SUT to monitor functional reserve consistently at the same time on different days. For example, an individual may perform these tests, with or without anaerobic capacity test, around breakfast time in the morning, or in the evening before bed time. Based on the test result, the individual may follow the customized guidance to preserve or reach his or her optimal functional state at the desired time, i.e., competition time.

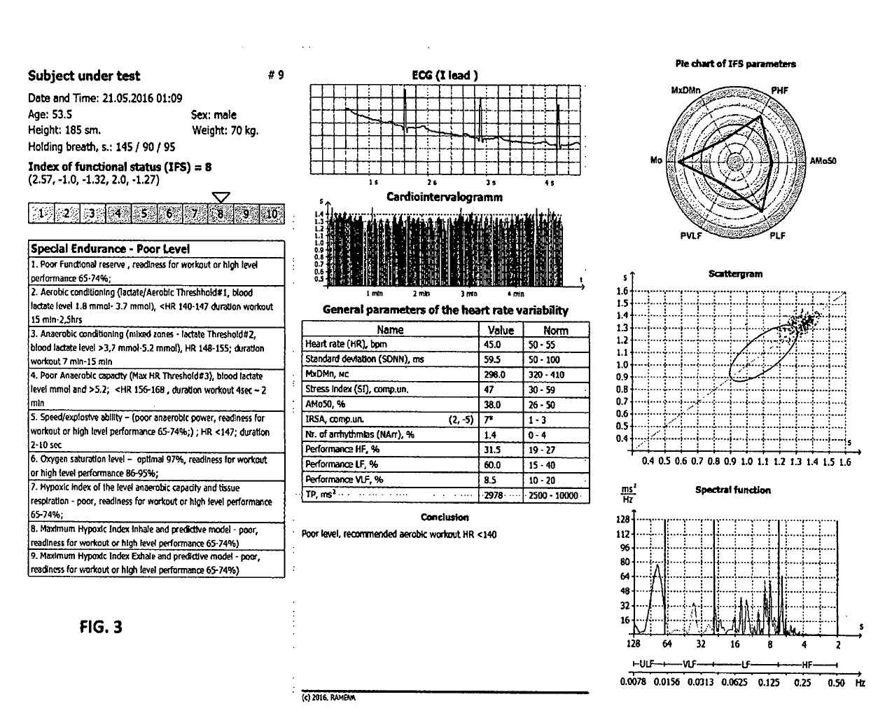

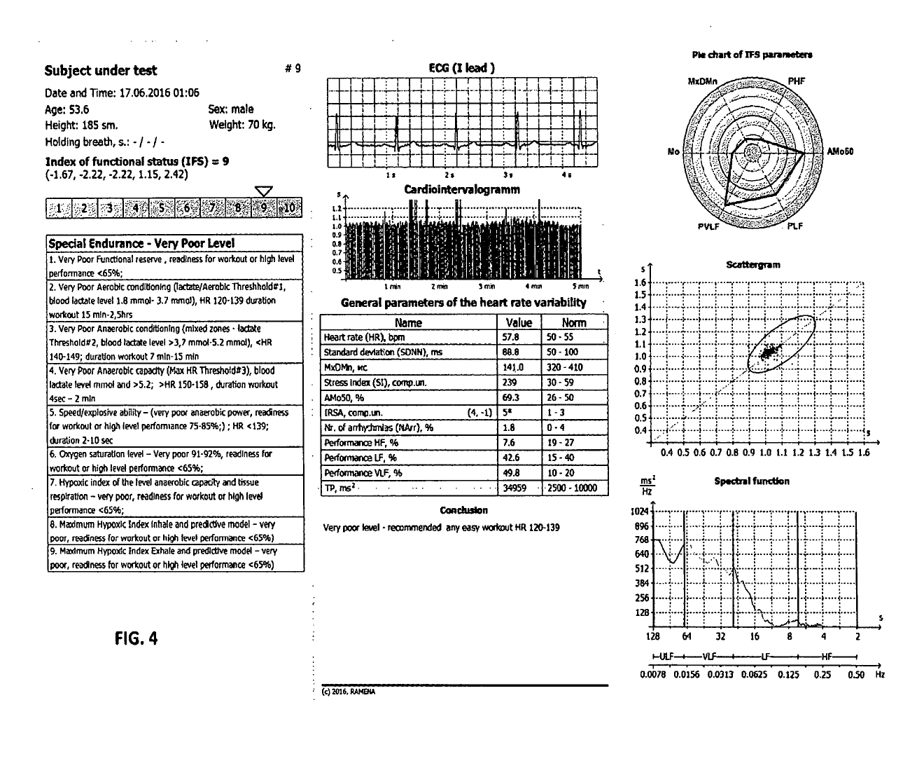

An HRV test is conducted to collect data from the sensors 101-104 which in turn is processed to produce a number of charts and graphics as well as textual output. FIG. 4 is a representative output of assessment results with only HMI test and oxygen saturation level test. FIG. 3 is a representative output of assessment results with HRV test, oxygen saturation level test, and anaerobic capacity test. The Index of Functional Status (IFS) shown in FIGS. 3 and 4 is an index that may scale between 1 and 10, visually represented by a color-coded spectrum using squares numbered 1 through 10. Squares numbered as IFS 1-3 are color-coded as the green zone. Squares numbered as IFS 4-7 are color-coded as the yellow zone. Squares numbered as IFS 8-10 are color-coded as the pink zone. The scale numbers and colors of the zones may be set differently to represent similar concept as shown in FIGS. 3 and 4. In the enablement represented by FIGS. 3 and 4, the higher IFS is, the poorer functional reserve, thus functional state, is. The green zone is deemed to be high or optimal level. The yellow zone is deemed to be moderate or working level. The pink zone is deemed to be poor or very poor level.

Referring to FIG. 3, the anaerobic capacity test is performed with all three phases, and the SUT's is 145 seconds, MHTAE is 90 seconds, and MHTIE is 95 seconds which includes 60 seconds while the SUT holds breath between inhalation and exhalation. The results also include graphical representations of the functional state of cardiac activities (cardiointervalogramm, scattergram, pie chart of IFS parameters, and spectral function). Data represented in the graphics is also represented in textual conclusions (shown in tables in the middle column and No. 6-9 on the left). The assessment logic generates the customized guidance No. 2-5 using the data propagated by the data collector 110 and by categorizing the SUT into one of four fitness levels: overweight, normal weight, active normal weight, and athletic.

As shown in FIGS. 3 and 4, the customized guidance for the same SUT is different in details. In FIG. 3, the SUT performed anaerobic capacity test, and the IFS is 8. In FIG. 4 where the SUT had been travelling right before the assessment, and the SUT did not perform an anaerobic capacity test, the IFS is 9. Although human bodies' functional status fluctuates from time to time, the SUT's travel right, before the assessment in FIG. 4 was a factor to result in a higher IFS (lower functional reserve) because the SUT was unable to maintain a consistent and routine breathing exercise that resembles the anaerobic capacity test while travelling. FIGS. 3 and 4 recommend the same customized exercises (No. 2-5) with the difference in heart rate requirement to improve functional reserve. No. 2 in FIG. 3 recommends the SUT to engage in aerobic exercises (some examples are walking, swimming, cycling) for anywhere between 15 minutes and 2½ hours while maintaining heart rate between 140 and 147. No. 2 in FIG. 4 recommends the same but heart rate needs to be in the range of 120-139 while engaging in aerobic exercises. The customized guidance No. 3 in both FIGS. 3 and 4 recommends the SUT to engage in anaerobic. exercises (examples include weight lifting, sprinting, and jumping) for anywhere between 7 minutes and 15 minutes, with the difference again being maintaining heart rate between 148 and 155 in FIG. 3 and between 140 and 149 in FIG. 4. Whether or not the SUT finds it challenging to engage in anaerobic exercises for as long as 7 minutes as recommended in No. 3, the SUT can choose the suggested anaerobic exercise in No. 4 to work out between 4 seconds and 2 minutes while maintaining heart rate either between 156 and 168 (FIG. 3) or between 150 and 158 (FIG. 4). Similarly, the recommended exercise to improve speed and explosive ability in No. 5 is for the SUT to engage in exercises between 2 seconds and 10 seconds while maintaining heart rate no higher than 147 (FIG. 3) or no higher than 139 (FIG. 4). Such exercises may be sprint running and weight lifting.

No. 6 in the assessment output displays oxygen saturation level result based on oxygen saturation level test.

No. 7 through 9 are assessment results of the SUT's anaerobic capacity, which may be generated without actually performing the anaerobic capacity test or with fewer phases of the anaerobic capacity test. Data from HRV test alone may produce results for No. 7 through 9 because an individual's heart indicates the integrity of the entire human body. The lack of anaerobic capacity test or the reduced number of phases of anaerobic capacity test merely reduces the SUT's opportunity to improve his or her functional reserve but does not jeopardize the assessment results. However, unless the SUP engages in anaerobic exercises in a manner recommended by the anaerobic capacity test, consistent failure to perform anaerobic capacity test or perform fewer than all three phases of anaerobic capacity test will result in reduced functional reserve over time, thus a higher IFS as represented in the enablement shown in FIGS. 3 and 4.

It is noted that, as used in this specification and the appended claims, the singular forms “a,” “an,” and “the,” include plural referents unless expressly and unequivocally limited to one referent, As used herein, the term “include” and its grammatical variants are intended to be non-limiting, such that recitation of items in a list is not to the exclusion of other like items that can be substituted or other items that can be added to the listed items.

Upon studying the disclosure, it will be apparent to those skilled in the art. Mat various modifications and variations can be made in the invention and methods of various embodiments of the invention. Other embodiments will be apparent to those skilled in the art from consideration of the specification and practice of the embodiments disclosed herein. It is intended that the specification be considered as examples only. The various embodiments are not necessarily mutually exclusive, as some embodiments can be combined with one or more other embodiments to form new embodiments.

What is claimed is:

1. A method of monitoring, assessing and managing functional state of a subject under test comprising the steps:
a) collecting a set of data from a group of physical state tests performed on the subject under test, including:
i) heart rate variability test,
ii) oxygen saturation level test; and
iii) anaerobic capacity test by measuring length of time passed while holding breath;
b) propagating the set of data into a processing computer;
c) assessing the set of data at the processing computer using assessment logic; and
d) displaying assessment output.

2. The method of claim 1, wherein the assessment output includes customized guidance to increase functional state.

3. The method of claim 1, wherein the group of physical state tests are all performed concurrently.

4. The method of claim 1, wherein the anaerobic capacity test is performed separately at a different time.

5. The method of claim 1, wherein the anaerobic capacity test is performed while the subject under test is at rest.

6. The method of claim 1, wherein the anaerobic capacity test is performed while the subject under test is engaging in physical activity.

7. The method of claim 1, therein the anaerobic capacity test is performed to measure maximum length of time passed while the subject under test holds breath beginning immediately following a completion of inhalation.

8. The method of claim 1, wherein the anaerobic capacity test is performed to measure maximum length of time passed while the subject under test holds breath beginning immediately following a completion of exhalation.

9. The method of claim 1, wherein the anaerobic capacity test is performed to measure maximum length of time passed for a complete breathing cycle including inhalation and exhalation.

10. The method of claim 9, wherein the breathing cycle includes time passed while the subject under test holds breath between inhalation and exhalation.

11. The method of claim 1, wherein the subject under test takes several deep cycles of inhalation and exhalation before the anaerobic capacity test is performed.

12. The method of claim 1, wherein the anaerobic capacity test is performed at a fixed time consistently on different days.

13. The method of claim 1, wherein the step of propagating the set of data is via a USB cable connection.

14. The method of claim 1, wherein the step of propagating the set of data is via wireless network.

15. An apparatus comprising:
a) a data collector having a plurality of sensors for physical state tests including heart rate variability and oxygen saturation level, wherein the data collector receives data from the plurality of sensors; and
b) a non-transitory computer-readable medium containing instructions to cause a programmable processor to perform operations comprising:
i) reading data from the data collector;
ii) receiving data from an anaerobic capacity test;
iii) processing data from the data collector and data from the anaerobic capacity test; and
iv) displaying a plurality of graphical and textual output on a user interface.

16. The apparatus of claim 15, wherein the data collector is battery-powered.

17. The apparatus of claim 15, wherein the data collector amplifies, filters and digitizes analog signals from the plurality of sensors.

18. The apparatus of claim 15, wherein the data collector is connected to the computer-readable medium via a USB cable.

19. The apparatus of claim 15, wherein the data collector is connected to the computer-readable medium via wireless network.

20. The apparatus of claim 15, wherein the plurality of sensors are connected to the data collector via a data cable.
